(19) United States
(12) Patent Application Publication
(10) Pub. No.: US 2003/0003492 A1
(43) Pub. Date: Jan. 2, 2003

(54) Colorimetric nanocrystal sensors, methods of making, and use thereof

(76) Inventors: Benjamin L. Miller, Rochester, NY (US); Todd D. Krauss, Penfield, NY (US)

Correspondence Address: Michael L. Goldman, NIXON PEABODY LLP, Clinton Square, P.O. Box 31051, Rochester, NY, 14603-1051, US

(21) Appl. No.: 10/171,136

(22) Filed: Jun. 13, 2002

Related U.S. Application Data
(60) Provisional application No. 60/297,868, filed on Jun. 13, 2001.

(51) Int. Cl. C12Q 1/68; G01N 33/554; G01N 33/569; G01N 33/53; G01N 33/542; C12M 1/34; B32B 5/16

(52) U.S. Cl. 435/6; 435/7.9; 435/287.2; 428/404

(57) ABSTRACT

The present invention relates to one or more different types of calorimetric sensor agents and one or more different types of fluorescence quenching agents or substrates, as well as their combination and various uses thereof, including detection of target molecules in a sample.

This application claims the benefit of U.S. Provisional Patent Application Serial No. 60/297,868, filed Jun. 13, 2001, which is hereby incorporated by reference in its entirety.

### FIELD OF THE INVENTION

The present invention relates to semiconductor nanocrystals functionalized with a non-biopolymer ligand or a ligand-binding molecule to form a colorimetric sensor agent, the combination thereof with quenching agents that interfere with nanocrystal fluorescence, and their use for detecting various target molecules in a sample.

### BACKGROUND OF THE INVENTION

Ever increasing attention is being paid to detection and analysis of low concentrations of analytes in various biologic and organic environments. Qualitative analysis of such analytes is generally limited to the higher concentration levels, whereas quantitative analysis usually requires labeling with a radioisotope or fluorescent reagent. Such procedures are time consuming and inconvenient. Thus, it would be extremely beneficial to have a quick and simple means of qualitatively and quantitatively detecting analytes at low concentration levels.

Solid-state sensors and particularly biosensors have received considerable attention lately due to their increasing utility in chemical, biological, and pharmaceutical research as well as disease diagnostics. In general, biosensors consist of two components: a highly specific recognition element and a transducing structure that converts the molecular recognition event into a quantifiable signal. Biosensors have been developed to detect a variety of biomolecular complexes including oligonucleotide pairs, antibody-antigen, hormone-receptor, enzyme-substrate and lectin-glycoprotein interactions. Signal transductions are generally accomplished with electrochemical, field-effect transistor, optical absorption, fluorescence or interferometric devices.

It is known that semiconductor and metal oxide nanocrystals (also known as quantum dots) can be used as biosensors. For example, U.S. Pat. No. 6,333,110 to Barbera-Guillem describes a semiconductor nanocrystal particle which is water soluble and has been functionalized with an affinity ligand that has binding specificity and avidity for a molecular component of, or associated with, a substrate. Similar semiconductor nanocrystals and their use in biological detection schemes have also been described in U.S. Pat. No. 6,274,323 to Bruchez et al. and U.S. Pat. No. 6,306,610 to Bawendi et al.

It would be desirable to provide a system for using functionalized semiconductor nanocrystals in biological detection systems, either in tissue or in aqueous solutions or suspensions, whereby fluorescent emissions of the semiconductor nanocrystals are readily quenched or shifted prior to binding of a ligand to a biological target.

The present invention is directed to overcoming these and other deficiencies in the art.

### SUMMARY OF THE INVENTION

A first aspect of the present invention relates to a colorimetric sensor agent that includes: a nanocrystal particle including a semiconductor material and a non-biopolymer ligand bound to the nanocrystal particle, the non-biopolymer ligand including a target molecule-binding moiety.

A second aspect of the present invention relates to the combination of the colorimetric sensor agent according to the first aspect of the invention and a quenching agent that includes (i) a metal particle or fluorophore and (ii) a ligand-binding moiety bound to the target molecule-binding moiety of the non-biopolymer ligand, wherein the metal particle or fluorophore absorbs fluorescent emissions from the semiconductor material while the quenching agent remains bound to the colorimetric sensor agent.

A third aspect of the present invention relates to a colorimetric sensor agent that includes: a nanocrystal particle including a semiconductor material and a ligand-binding moiety that binds to a target molecule-binding moiety of a non-biopolymer ligand, wherein the nanocrystal particle fluoresces upon dissociation of the ligand-binding moiety from a non-biopolymer ligand that is bound to a substrate that quenches fluorescence thereof.

A fourth aspect of the present invention relates to the combination of the colorimetric sensor agent according to the third aspect of the invention, a substrate that quenches fluorescent emissions and a non-biopolymer ligand bound to the substrate, the non-biopolymer ligand including a target molecule binding moiety, wherein upon dissociation of the ligand-binding moiety from the target molecule-binding moiety of the non-biopolymer ligand, quenching of fluorescent emissions from the semiconductor material by the substrate diminishes.

A fifth aspect of the present invention relates to a substrate having a surface to which is bound a colorimetric sensor according to the first aspect of the invention.

A sixth aspect of the invention relates to a method of making a colorimetric sensor agent, the method including: reacting a non-biopolymer ligand that includes a target molecule-binding moiety and a nanocrystal-binding moiety with a nanocrystal particle that includes a semiconductor material or a metal, the reacting being performed under conditions effective to bind the non-biopolymer ligand to the semiconductor material or the metal, thereby forming the colorimetric sensor agent.

A seventh aspect of the present invention relates to a method of making a colorimetric sensor agent according to the third aspect of the invention, the method including: reacting a compound that includes a ligand-binding moiety and a nanocrystal-binding moiety with a nanocrystal particle that includes a semiconductor material, the reacting being performed under conditions effective to bind the compound to the semiconductor material, thereby forming the colorimetric sensor agent.

An eighth aspect of the present invention relates to a method of detecting for the presence of a target molecule in a sample, the method including: introducing a sample to a solution or suspension that includes a plurality of colorimetric sensor agents according to the first aspect of the invention; rinsing unbound colorimetric sensor agents from the sample; and determining whether the rinsed sample fluoresces, indicating the presence of a target molecule in the sample.

A ninth aspect of the present invention relates to a method of detecting for the presence of a target molecule in a sample, the method including: introducing a sample to a solution or suspension that includes a plurality of the colorimetric sensor agents-quenching agent combinations in accordance with the second aspect of the invention; and determining whether the solution or suspension changes color after said introducing, wherein a color or fluorescent emission change indicates the presence of a target molecule in the sample.

A tenth aspect of the present invention relates to a method of detecting for the presence of a target molecule in a sample, the method including: introducing a sample to a solution or suspension which includes the combination of the substrate to which is bound the non-biopolymer ligand and the colorimetric sensor agent, in accordance with the fourth aspect of the invention; and determining whether the solution or suspension changes color after said introducing, wherein a color or fluorescent emission change indicates the presence of a target molecule in the sample.

An eleventh aspect of the present invention relates to a method of detecting for the presence of lipid A in a sample, the method including: introducing a sample to a solution or suspension comprising a plurality of colorimetric sensor agents according to the first aspect of the present invention, wherein the non-biopolymer ligand is a peptidomimetic compound having lipid A binding activity; rinsing unbound colorimetric sensor agents from the sample; and determining whether the rinsed sample fluoresces, indicating the presence of lipid A in the sample.

A twelfth aspect of the present invention relates to a method of detecting for the presence of lipid A in a sample, the method including: introducing a sample to a solution or suspension that includes a plurality of the colorimetric sensor agents-quenching agent combinations according to the second aspect of the present invention, wherein the non-biopolymer ligand is a peptidomimetic compound having lipid A binding activity; and determining whether the solution or suspension changes color after said introducing, wherein a color or fluorescent emission change indicates the presence of lipid A in the sample.

A thirteenth aspect of the present invention relates to a method of detecting for the presence of lipid A in a sample, the method including: introducing a sample to a solution or suspension which includes the combination of the substrate to which is bound the non-biopolymer ligand and the colorimetric sensor agent, in accordance with the fourth aspect of the invention, wherein the non-biopolymer ligand is a peptidomimetic compound having lipid A binding activity; and determining whether the solution or suspension changes color after said introducing, wherein a color or fluorescent emission change indicates the presence of lipid A in the sample.

A fourteenth aspect of the present invention relates to a method of detecting for the presence of Gram negative bacteria in a sample, the method including: introducing a sample to a solution or suspension comprising a plurality of colorimetric sensor agents according to the first aspect of the invention, wherein the non-biopolymer ligand is a peptidomimetic compound having lipid A binding activity; rinsing unbound colorimetric sensor agents from the sample; and determining whether the rinsed sample fluoresces, indicating the presence of lipid A and therefore Gram negative bacteria in the sample.

A fifteenth aspect of the present invention relates to a method of detecting for the presence of Gram negative bacteria in a sample, the method including: introducing a sample to a solution or suspension comprising a plurality of the colorimetric sensor agents-quenching agent combinations according to the second aspect of the invention, wherein the non-biopolymer ligand is a peptidomimetic compound having lipid A binding activity; and determining whether the solution or suspension changes color after said introducing, wherein a color or fluorescent emission change indicates the presence of lipid A and therefore Gram negative bacteria in the sample.

A sixteenth aspect of the present invention relates to a method of detecting for the presence of Gram negative bacteria in a sample, the method including: introducing a sample to a solution or suspension which includes the combination of the substrate to which is bound the non-biopolymer ligand and the colorimetric sensor agent, in accordance with the fourth aspect of the invention, wherein the non-biopolymer ligand is a peptidomimetic compound having lipid A binding activity; and determining whether the solution or suspension changes color after said introducing, wherein a color or fluorescent emission change indicates the presence of lipid A and therefore Gram negative bacteria in the sample.

### BRIEF DESCRIPTION OF THE DRAWINGS

FIGS. 1A-B illustrate the relationship between a colorimetric sensor agent according to a first embodiment and a quenching agent of the present invention. In FIG. 1A, the ligand-binding moiety of a quenching agent is shown bound to the target molecule-binding moiety of the non-biopolymer ligand. As a result, fluorescence emissions by the nanocrystal particle are quenched or absorbed by the metal particle or fluorophore, which is located a distance d therefrom. In FIG. 1B, the quenching agent is shown to be displaced from the target molecule-binding moiety of the non-biopolymer ligand by a target molecule (i.e., due to differences in their affinities). As a result of such displacement, the metal particle or fluorophore of the quenching agent is greater than a distance d from the nanocrystal particle and fluorescence emissions therefrom can be detected.

FIGS. 2A-B illustrate the relationship between a colorimetric sensor agent according to a second embodiment and a quenching substrate of the present invention. In FIG. 2A, the ligand-binding moiety of a colorimetric sensor agent is shown bound to the target molecule-binding moiety of the non-biopolymer ligand, which itself is tethered to a quenching substrate. As a result, fluorescence emissions by the nanocrystal particle are quenched or absorbed by the substrate, which is located a distance d therefrom. In FIG. 2B, the colorimetric sensor agent is shown to be displaced from the target molecule-binding moiety of the non-biopolymer ligand by a target molecule (i.e., due to differences in their affinities). As a result of such displacement, the colorimetric sensor agent is greater than a distance d from the quenching substrate and fluorescence emissions therefrom can be detected.

FIG. 3 illustrates equipment setup for detection of fluorescence emissions from nanocrystal particles.

FIGS. 4A-B illustrate spectroscopic results following the linkage of TWTCP to CdSe nanocrystal particles. FIG. 4A shows the emission of TWTCP alone. FIG. 4B shows the emission of TWTCP-functionalized CdSe nanocrystal particles.

FIG. 5 depicts schematically the detection of E. coli using a TWTCP-functionalized nanocrystal particle.

FIGS. 6A-B illustrate fluorescence and darkfield light-scattering images of TWTCP-functionalized nanocrystal particles following their exposure to E. coli as depicted in FIG. 5.

FIG. 7 illustrates an inert substrate to which is attached a colorimetric sensor according to a first embodiment of the present invention. Although not shown, the substrate bound colorimetric sensor agent can be used in combination with an appropriate quenching agent of the present invention.

### DETAILED DESCRIPTION OF THE INVENTION

The present invention relates to one or more different types of colorimetric sensor agents and one or more different types of fluorescence quenching agents, as well as their combination and various uses.

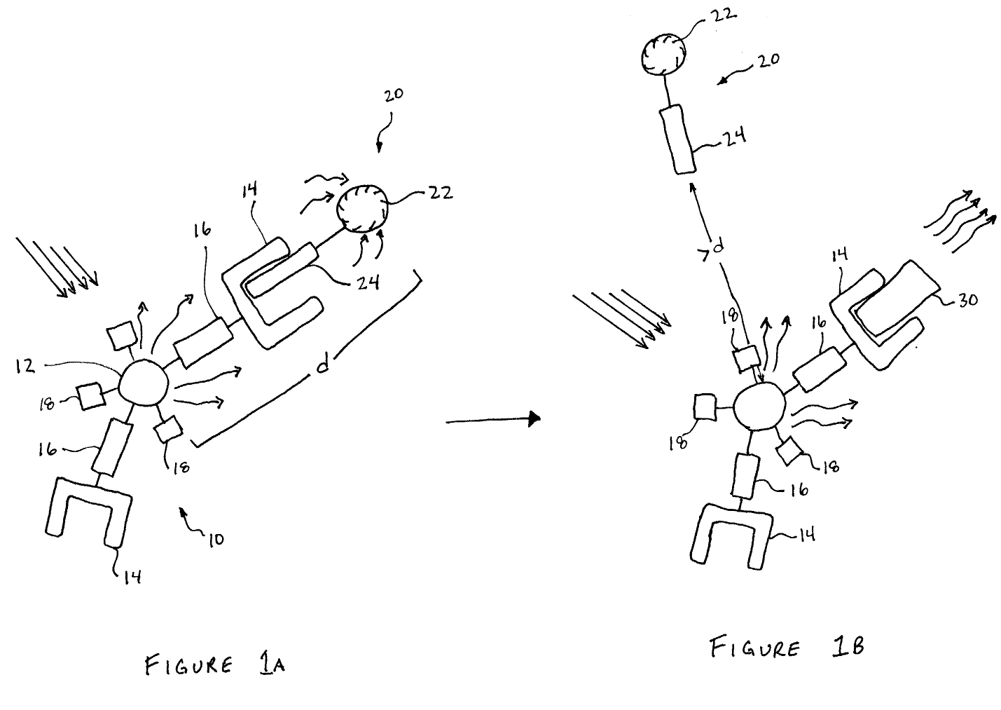

A first embodiment of colorimetric sensor agent and corresponding fluorescence quenching agent are shown in FIGS. 1A-B. The colorimetric sensor agent 10 includes a nanocrystal particle 12 formed of a semiconductor material and a non-biopolymer ligand 14 bound to the nanocrystal particle via a linker 16. Also shown are capping molecules 18 that increase the water-solubility of the nanocrystal particles. The capping molecule and the linker can be the same or different. The quenching agent 20 includes a metal particle or fluorophore 22 and a ligand-binding moiety 24 that is capable of binding to the target molecule-binding moiety of the non-biopolymer ligand.

As used herein, nanocrystal particles or semiconductor nanocrystals (also known as Quantum Dot™ particles), whose radii are smaller than the bulk exciton Bohr radius, constitute a class of materials intermediate between molecular and bulk forms of matter. Quantum confinement of both the electron and hole in all three dimensions leads to an increase in the effective band gap of the material with decreasing crystallite size. Consequently, both the optical absorption and emission of semiconductor nanocrystals shift to the blue (higher energies) as the size of the nanocrystals gets smaller.

The core of the nanocrystal particles is substantially monodisperse. By monodisperse, it is meant a colloidal system in which the suspended particles have substantially identical size and shape, i.e., deviating less than about 10% in rms diameter in the core, and preferably less than about 5% in the core.

Particles size can be between about 1 nm and about 1000 nm in diameter, preferably between about 2 nm and about 50 nm, more preferably about 5 nm to about 20 nm (such as about 5, 6, 7, 8, 9, 10, 11, 12, 13, 14, 15, 16, 17, 18, 19, or 20 nm).

When capped quantum dots of the invention are illuminated with a primary light source, a secondary emission of light occurs of a frequency that corresponds to the band gap of the semiconductor material used in the quantum dot. The band gap is a function of the size of the nanocrystal particle. As a result of the narrow size distribution of the capped nanocrystal particles, the illuminated Her nanocrystal particles emit light of a narrow spectral range resulting in high purity light. Spectral emissions in a narrow range of no greater than about 60 nm, preferably no greater than about 40 nm and most preferably no greater than about 30 nm at full width half max (FWHM) are observed. Spectral emissions in even narrower ranges are most preferred.

The nanocrystal particles are preferably passivated or capped either with organic or inorganic passivating agents to eliminate energy levels at the surface of the crystalline material that lie within the energetically forbidden gap of the bulk interior. These surface energy states act as traps for electrons and holes that would normally degrade the luminescence properties of the material. Such passivation produces an atomically abrupt increase in the chemical potential at the interface of the semiconductor and passivating layer (Alivisatos, J. Phys. Chem. 100:13226 (1996), which is hereby incorporated by reference in its entirety). As a result, higher quantum efficiencies can be achieved.

Exemplary capping agents include organic moieties such as tri-n-octyl phosphine (TOP) and tri-n-octyl phosphine oxide (TOPO) (Murray et al., J. Am. Chem. Soc. 115:8706 (1993); Kuno et al., J. Phys. Chem. 106(23):9869 (1997), each of which is hereby incorporated by reference in its entirety), as well as inorganic moieties such as CdS-capped CdSe and the inverse structure (Than et al., J. Phys. Chem. 100:8927 (1996), which is hereby incorporated by reference in its entirety), ZnS grown on CdS (Youn et al., J. Phys. Chem. 92:6320 (1988), which is hereby incorporated by reference in its entirety), ZnS on CdSe and the inverse structure (Kortan et al., J. Am. Chem. Soc. 112:1327 (1990); Hines et al., J. Phys. Chem. 100:468 (1996), each of which is hereby incorporated by reference in its entirety), ZnSe-capped CdSe nanocrystals (Danek et al., Chem. Materials 8:173 (1996), which is hereby incorporated by reference in its entirety), and SiO₂ on Si (Wilson et al., Science 262:1242 (1993), which is hereby incorporated by reference in its entirety).

In general, particles passivated with an inorganic coating are more robust than organically passivated particles and have greater tolerance to processing conditions necessary for their incorporation into devices. Particles that include a “core” of one or more first semiconductor materials can be surrounded by a “shell” of a second semiconductor material.

Thus, the nanocrystal particles as used in the present invention can be formed of one or more metals or one or more semiconducting materials. Suitable metals include, without limitation, gold, silver, platinum, copper, cobalt, iron, iron-platinum, etc. Suitable semiconducting materials include, without limitation, a group IV material alone (e.g., Si and Ge), a combination of a group IV material and a group VI material, a combination of a group III material and a group V material, or a group II material and a group VI material. When a combination of materials are used, the semiconducting materials are presented in a “core/shell” arrangement.

Suitable core/shell material combinations include, without limitation, group IV material forming the core and group VI materials forming the shell; group III material forming the core and group V materials forming the shell; and group II material forming the core and group VI materials forming the shell. Exemplary core/shell combinations for groups IV/VI are: Pb and one or more of S, Se, and Te. Exemplary core/shell combinations for groups III/V are: one or more of Ga, In, and Al as the group III material and one or more of N, P, As, and Sb as the group V material. Exemplary core/shell combinations for groups II/VI are: one or more of Cd, Zn, and Hg as the group II material, and one or more of S, Se, and Te as the group VI material. Other combinations now known or hereinafter developed can also be used in the present invention.

Fluorescent emissions of the resulting nanocrystal particles can be controlled based on the selection of materials and controlling the size distribution of the particles. For example, ZnSe and ZnS particles exhibit fluorescent emission in the blue or ultraviolet range (˜400 nm or less); Au, Ag, CdSe, CdS, and CdTe exhibit fluorescent emission in the visible spectrum (between about 440 and about 700 nm); InAs and GaAs exhibit fluorescent emission in the near infrared range (˜1000 nm), and PbS, PbSe, and PbTe exhibit fluorescent emission in the near infrared range (i.e., between about 700-2500 nm). By controlling growth of the nanocrystal particles it is possible to produce particles that will fluoresce at desired wavelengths. As noted above, smaller particles will afford a shift to the blue (higher energies) as compared to larger particles of the same material(s).

Preparation of the nanocrystal particles can be carried out according to known procedures, e.g., Murray et al., MRS Bulletin 26(12):985-991 (2001); Murray et al., IBM J. Res. Dev. 45(1):47-56 (2001); Sun et al., J. Appl. Phys. 85(8, Pt. 2A): 4325-4330 (1999); Peng et al., J. Am. Chem. Soc. 124(13):3343-3353 (2002); Peng et al., J. Am. Chem. Soc. 124(9):2049-2055 (2002); Qu et al., Nano Lett. 1(6):333-337 (2001); Peng et al., Nature 404(6773):59-61 (2000); Talapin et al., J. Am. Chem. Soc. 124(20):5782-5790 (2002); Shevenko et al., Advanced Materials 14(4):287-290 (2002); Talapin et al., Colloids and Surfaces, A: Physiochemical and Engineering Aspects 202(2-3):145-154 (2002); Talapin et al., Nano Lett. 1(4):207-211 (2001), each of which is hereby incorporated by reference in its entirety.

Whether in a core/shell arrangement or otherwise passivated with other compounds, the nanocrystal particles can also be rendered water soluble, which is desirable when the particles intend to be utilized in a biological detection system. To make water-soluble nanocrystal particles, hydrophilic capping compounds are bound to the particles. One suitable class includes carboxylic acid capping compounds with a thiol functional group (forming a sulfide bridge with the nanocrystal particle), which can be reacted with the nanocrystal. Exemplary capping compounds include, without limitation, mercaptocarboxylic acid, mercaptofunctionalized amines (e.g., aminoethanethiol-HCl, homocysteine, or 1-amino-2-methyl-2-propanethiol-HCl), mercaptofunctionalized sulfonates, mercaptofunctionalized alkoxides, mercaptofunctionalized phosphates and phosphonates, aminofunctionalized sulfonates, aminofunctionalized alkoxides, amino functionalized phosphates and phosphonates, phosphine(oxide)functionalized sulfonates, phosphine(oxide)functionalized alkoxides, phosphine(oxide)functionalized phosphates and phosphonates, and combinations thereof. Procedures for binding these capping compounds to the nanocrystal particles are known in the art, e.g., U.S. Pat. No. 6,319,426 to Bawendi et al., which is hereby incorporated by reference in its entirety.

As used herein, a non-biopolymer ligand is meant to include any ligand provided that the ligand is not a nucleic acid molecule, either DNA or RNA. For purposes of the present invention, DNA and RNA molecules are compounds that consist solely of two or more nucleotides linked by phosphodiester bonds. Suitable non-biopolymer ligands can be any compound that binds to a target molecule via its target-molecule binding moiety (i.e., a functional portion of the molecule which is the active site for binding to the target). Exemplary target molecules include, without limitation, receptor molecules, preferably a biological receptor molecule such as a protein, RNA molecule, or DNA molecule. In practice, the target molecule is one which is associated with a particular disease state, a particular pathogen, etc. Such target molecules, when identified in a sample, indicate the presence of a pathogen or the existence of a disease state (or potential disease state).

Preferably, the ligand can be either a small molecule, a carbohydrate, or a protein or polypeptide.

Exemplary small molecules include, without limitation: avidin, peptidomimetic compounds, and vancomycin. A number of peptidomimetic compounds are disclosed in U.S. patent application Ser. No. 09/568,403 to Miller et al., filed May 10, 2000, which is hereby incorporated herein by reference in its entirety. A preferred peptidomimetic compound that is known to bind lipopolysaccharide (lipid A) is a tetratryptophan ter-cyclopentane (TWTCP). Other peptidomimetic compounds can also be employed. Other small molecule, non-biopolymer ligands are disclosed in U.S. patent application Ser. No. 09/181,108 to Miller et al., filed Oct. 28, 1998; and U.S. patent application Ser. No. 09/838,971 to Miller et al., filed Apr. 20, 2001, each of which is hereby incorporated by reference in its entirety.

Exemplary proteins or polypeptides include, without limitation, a receptor for cell surface molecule or fragment thereof; a lipid A receptor; an antibody or fragment thereof; peptide monobodies of the type disclosed in U.S. patent application Ser. No. 09/096,749 to Koide, filed Jun. 12, 1998, and U.S. patent application Ser. No. 10/006,760 to Koide, filed Nov. 19, 2001, each of which is hereby incorporated by reference in its entirety; a lipopolysaccharide-binding polypeptide; a peptidoglycan-binding polypeptide; a carbohydrate-binding polypeptide; a phosphate-binding polypeptide; a nucleic acid-binding polypeptide; and polypeptides which bind organic warfare agents such as tabun, sarin, soman, GF, VX, mustard agents, botulinium toxin, Staphylococcus entertoxin B, and saitotoxin.

Coupling of the non-biopolymer ligand to the nanocrystal particles can be achieved using one or more known coupling procedures by derivatizing the non-biopolymer ligand with a linker group or molecule for coupling to the nanocrystal particle or by derivatizing the nanocrystal particle with a linker group or molecule for coupling to a non-biopolymer ligand. Of these approaches, the former is preferred because it allows better stoichiometric control of the ratio of non-biopolymer ligands to capping agents on a given particle.

The linker group or molecule refers to a compound or molecule that acts as a molecular bridge to operably link together two different molecules, with one portion of the linker group or molecule being linked to the non-biopolymer ligand and another portion of the linker group or molecule being linked to the nanocrystal particle. The linker group or molecule can be linked to the two components via a step-wise reaction (e.g., first to the non-biopolymer ligand and then to the nanocrystal or vice versa). The linker can be a homo-bifunctional linker or a hetero-bifunctional linker.

Regardless of the procedures employed, the non-biopolymer ligand becomes bound or operably linked to the nanocrystal particle. It is intended that this bond or fusion thus formed is the type of association which is sufficiently stable so that it is capable of withstanding the conditions or environments encountered during use thereof, i.e., in detection procedures. Preferably, the bond is a covalent bond, although other types of stable bonds can also be formed.

As described more fully in the examples, the process involves reacting a non-biopolymer ligand that includes a target molecule-binding moiety and a nanocrystal-binding moiety (e.g., thiol, amino group, carboxylic acid, phosphine, or phosphine oxide) with a nanocrystal particle comprising a semiconductor material or a metal under conditions effective to bind the non-biopolymer ligand to the semiconductor material or metal via the nanocrystal-binding moiety. The reaction conditions for binding these non-biopolymer ligands to the nanocrystal are known in the art (e.g., U.S. Pat. No. 6,319,426 to Bawendi et al., which is hereby incorporated by reference in its entirety), allowing formation of a covalent bond between the nanocrystal-binding moiety of the non-biopolymer ligand and the semiconductor material or metal, i.e., forming a sulfide bridge, amido bridge, phosphine or phosphine oxide bridge, carboxylate bridge.

To prepare the non-biopolymer ligand that has been functionalized with a nanocrystal-binding moiety, the non-biopolymer ligand precursor is preferably reacted with a capping molecule of the type described above (i.e., having a nanocrystal-binding moiety) under conditions effective to form the non-biopolymer ligand.

In addition, the nanocrystal particle can also be functionalized with a capping molecule of the type described above, e.g., hydrophilic capping compounds. This reaction can be carried out after binding of the non-biopolymer ligand or, more preferably, simultaneously therewith. The capping molecule used to render the nanocrystal particle water-soluble can be the same capping molecule used to prepare the non-biopolymer ligand that is reacted with the nanocrystal particle to form the functional colorimetric sensor agent.

The quenching agent is formed of a metal or other fluorophore that is capable of quenching or absorbing the fluorescent emissions of the nanocrystal particle within the desired bandwidth.

Metals, when employed, offer the ability to completely or nearly completely quench the fluorescence emissions of the nanocrystal particle. Thus, no detectable emission peak will be detected while the quenching agent remains bound to the colorimetric sensor agent. Suitable metals include, without limitation, gold, silver, platinum, copper, cobalt, iron, iron-platinum, etc. Of these, gold, silver, and platinum are typically preferred.

Other fluorophores, when employed, offer the ability to shift (i.e., red-shift or blue-shift) the emission spectra. Thus, a first emission peak will be visible while the quenching agent remains bound to the colorimetric sensor agent and a second emission peak will be visible once the quenching agent has been displaced from the colorimetric sensor agent by the target. Suitable fluorophores include, without limitation, other nanocrystals of the type described above (i.e., having a different size or formed of different materials), fluorescent dyes, fluorescent polymers, or fluorescent proteins (e.g., green fluorescent proteins). The fluorophore of the quenching agent can be either a donor fluorophore or an acceptor fluorophore, in which case either an increase or a decrease, respectively, in fluorescent emission by the colorimetric sensor agent can be detected.

Operably linked to the metal particle or fluorophore is a ligand-binding moiety that is capable of binding to the target molecule-binding moiety of the non-biopolymer ligand. The ligand-binding moiety preferably has an affinity for the target molecule-binding moiety which is less than the affinity of the target molecule-binding moiety for its intended target. Thus, a ligand-binding moiety that is bound to the target molecule-binding moiety will be favorably displaced in the presence of the intended target. Suitable ligand-binding moieties include, without limitation, peptides, carbohydrates (e.g., sugars such as glucosamine), haptens, RNA aptamers, etc.

Coupling of the ligand-binding moiety to the metal particle or fluorophore can be achieved using one or more known coupling procedures, by derivatizing the ligand-binding moiety with a linker group or molecule for coupling to the metal particle or fluorophore as well as by derivatizing the metal particle or fluorophore with a linker group or molecule for coupling to a ligand-binding moiety. The linker group can generally be of the same type as described above for linking together the non-biopolymer ligand and the nanocrystal particle, in which case the same known chemistry can be employed. According to one approach, the procedures of Lin et al. (J. Am. Chem. Soc. 124:3508-3509 (2002), which is hereby incorporated by reference in its entirety) can be employed to form a thio-derivative of a sugar that is then attached to a gold particle.

Once the functionalized nanocrystal particles (colorimetric sensor agents) and corresponding quenching agents have been prepared, they are capable of use together to identify the presence of a target in a sample. The sample can be either a tissue sample in solid form or in fluid form. The sample can also be present in an aqueous solution.

As illustrated in FIGS. 1A-1B, the ligand-binding moiety 24 of a quenching agent 20 is shown bound to the target molecule-binding moiety of the non-biopolymer ligand 14. As a result, fluorescence emissions by the nanocrystal particle 12 are quenched or absorbed by the metal particle of fluorophore 22, which is located a distance d therefrom. In FIG. 1B, the quenching agent 20 is shown to be displaced from the target molecule-binding moiety of the non-biopolymer ligand 14 by a target molecule 30 (i.e., due to differences in their affinities). As a result of such displacement, the metal particle or fluorophore 22 is greater than a distance d from the nanocrystal particle 12 and fluorescence emissions therefrom can be detected.

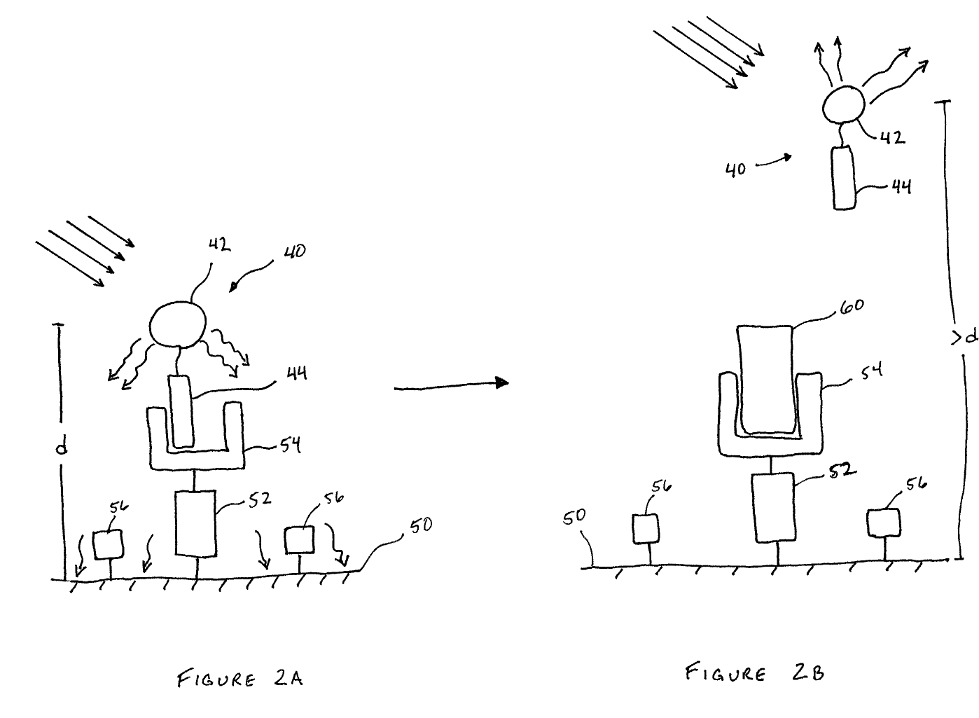

According to a second embodiment of the present invention is a colorimetric sensor agent and corresponding fluorescence quenching substrate as shown in FIGS. 2A-B. The colorimetric sensor agent 40 includes a nanocrystal particle 42 formed of a semiconductor material and a ligand-binding moiety 42 attached thereto. Attached to a quenching substrate 50 via a linker 52 is a non-biopolymer ligand 54 that includes a target molecule-binding moiety. Also shown are capping compounds 56 to regulate the concentration of non-biopolymer ligand on the quenching substrate.

The linker can be coupled to the non-biopolymer ligand and the metal substrate in the same manner described above for attaching the linker and non-biopolymer ligand to the nanocrystal particle in the first embodiment. Likewise, the ligand-binding moiety of the colorimetric sensor can be coupled to the nanocrystal particle in the same manner as described above with respect to its coupling to the metal particle of the first embodiment.

As illustrated in FIGS. 2A-2B, the ligand-binding moiety 44 of a colorimetric sensor agent 40 is shown bound to the target molecule-binding moiety of the non-biopolymer ligand 54. As a result, fluorescence emissions by the nanocrystal particle 42 are quenched or absorbed by the metal substrate 50, which is located a distance d therefrom. In FIG. 2B, the ligand-binding moiety 44 is shown to be displaced from the target molecule-binding moiety of the non-biopolymer ligand 54 by a target molecule 60 (i.e., due to differences in their affinities). As a result of such displacement, the quenching metal substrate 50 is greater than a distance d from the nanocrystal particle 42 and fluorescence emissions therefrom can be detected.

Regardless of the embodiment, it is believed that the quenching or absorption of fluorescence emissions is a direct result of the proximity between the nanocrystal particle (of the colorimetric sensor agent) and the metal particle or fluorophore (of the quenching agent). It is believed that suitable quenching is achieved when the two particles (or particle and substrate) are within about 50 angstroms, more preferably within about 30 angstroms, even more preferably within about 25 angstroms from one another. In general, more complete quenching can be achieved the closer together the nanocrystal particle and metal particle (or fluorophore) are.

As an alternative embodiment, a colorimetric sensor agent can be in the form of a non-biopolymer ligand of the type described above which is bound to a metal particle of the type described above (preferably gold, silver, or platinum). When multiple colorimetric sensor agents of this type aggregate about a single multivalent biological target (i.e., cell or tissue), the aggregation thereof changes the color or fluorescent emission of the solution or suspension in which the sensor agents reside. Basically, the emission characteristics of the metal particles in aggregate behave as if they are a single large particle when the inter-particle distance is very small, thereby exhibiting a shift in the peak emission characteristics. The shift, if large enough, can be detected by the naked eye. Otherwise, detection equipment of the type described below can be employed.

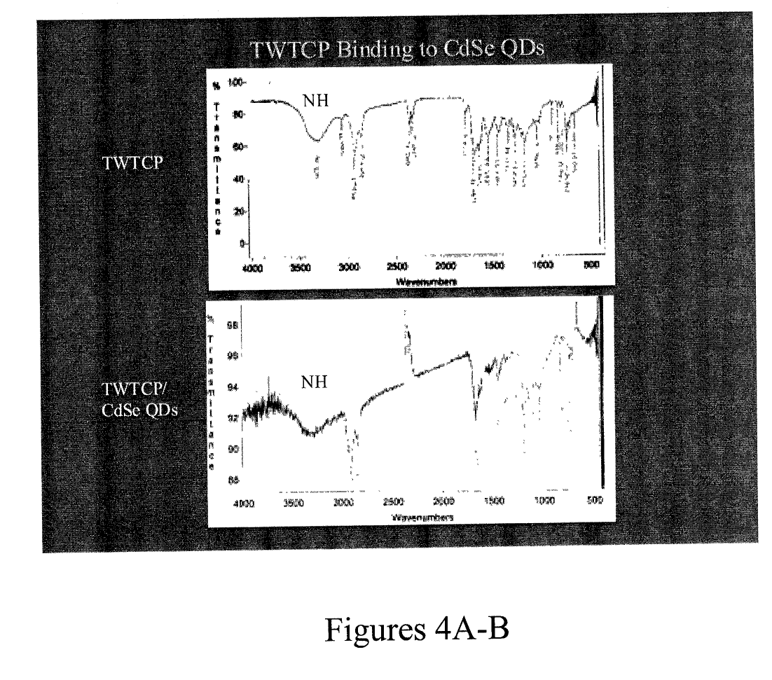

Detection of fluorescence emissions can be achieved using conventional detection equipment. Exemplary equipment are illustrated in FIG. 4. Briefly, a laser or other illumination source 100 passes its light to a sample stage 102, where the functionalized nanocrystal particles, sample, and any quenching agents or quenching substrate are present together. Any fluorescence illumination will be detected through a microscope 104 whose enhanced output is passed to a spectrometer 106 equipped with a detector 108. The spectrometer 106 and detector 108 are coupled to a printer 110 capable of printing a photographic image illustrating the fluorescent emission pattern emitted by the components present on the stage. Thus, the fluorescence emissions can be measured both before and after exposing a sample to the colorimetric sensor agents of the present invention.

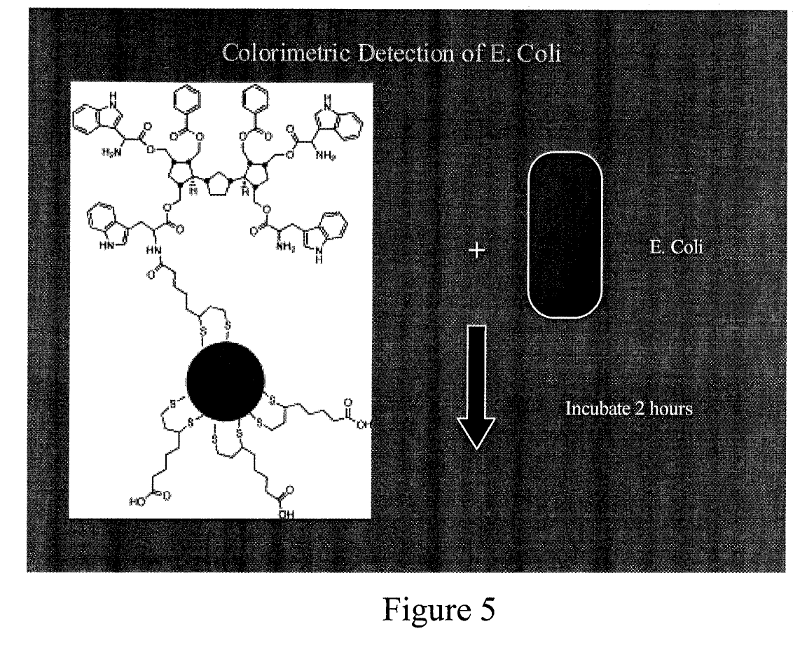

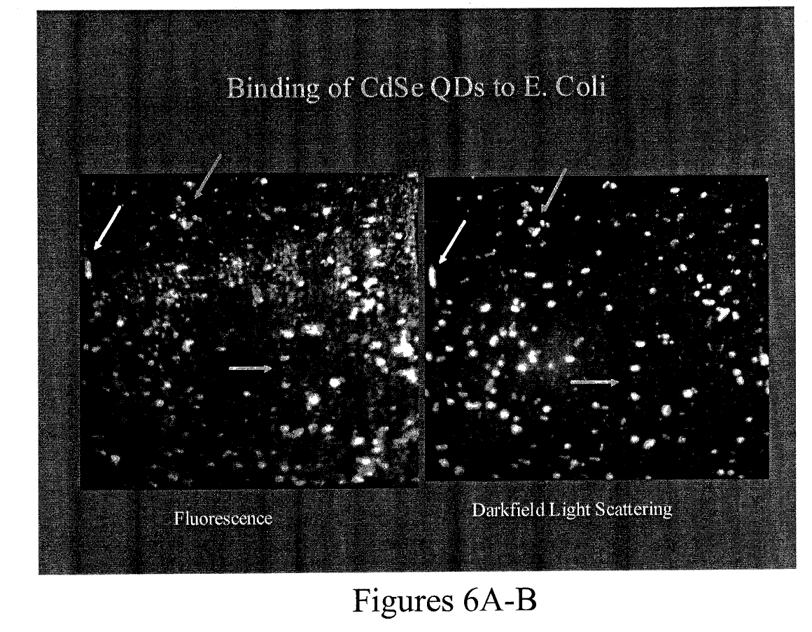

Certain peptidomimetic compounds as disclosed in U.S. patent application Ser. No. 09/568,403 to Miller et al., which is incorporated herein by reference in its entirety, are capable of binding to lipid A and, therefore, are useful for detecting not only the presence of lipid A in a sample, but also the presence of Gram negative bacteria in the sample. Basically, this is achieved by introducing a sample to a solution or suspension that includes a plurality of colorimetric sensor agents or the combination of colorimetric sensor agents and quenching agents or substrates in accordance with the present invention, where the non-biopolymer ligand is a peptidomimetic compound having lipid A binding activity. If desired of if necessary, colorimetric sensor agents that do not bind to the target can be removed from the sample, thereby minimizing any background fluorescence. Thereafter, a determination is made as to whether the sample fluoresces or whether the solution or suspension itself changes color after the introduction of the sample, where a color or fluorescent emission change indicates the presence of lipid A (and, thus, Gram negative bacteria) in the sample. FIGS. 5 and 6A-B illustrate how E. coli detection can be effected using the present invention.

Another aspect of the present invention relates to a substrate that has a surface to which is bound a colorimetric sensor agent in accordance with the first embodiment. Basically, the particle is coupled onto an inert solid substrate (i.e., ones that do not quench nanocrystal fluorescence) such as silica, or incorporated into a thin film. Coupling onto an inert solid substrate can be carried out as described above for metal substrate coupling or by using known silanization procedures.

Where the thin film is a polymeric thin film, the colorimetric sensor agents can be incorporated therein by binding to the nanocrystal particle a polymerization-reactive moiety (monomer or oligomer) that is capable of forming a co-polymer with a polymerizable composition, which includes oligomers, monomers, or combinations thereof. Suitable monomer and oligomer components include, without limitation, styrenes, divinylbenzenes, acrylates and methacrylates, acetylenes, alkylenes, olefins, etc. The formation of the polymeric thin-film can be carried out under standard polymerization conditions which are known in the art (see Ravve, Principles of Polymer Chemistry (2d edition), Kluwer Academic/Plenum Publishers, New York (2000), which is hereby incorporated by reference in its entirety. Moreover, incorporation of nanocrystal particles in such composite materials has been reported by Skaff et al. (J. Am. Chem. Soc. 124:5729-5733 (2002), which is hereby incorporated by reference in its entirety) using a cyclic olefin, which following ring opening can form polyolefin composite materials.

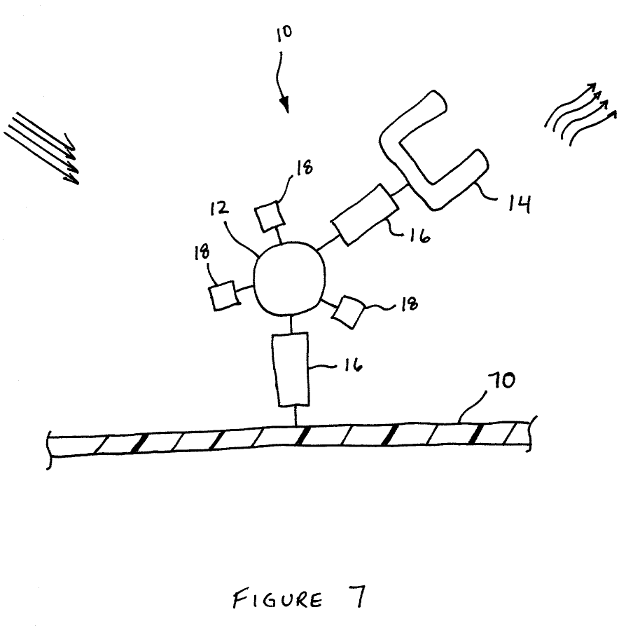

As shown in FIG. 7, a colorimetric sensor agent 10 is coupled to an inert substrate 70 and in the absence of a quenching agent 20 bound to the non-biopolymer ligand 14, fluorescence of the nanocrystal particle 14 can be detected.

Under carefully controlled conditions, it is expected to achieve detection sensitivities approaching the single nanocrystal level (Nirmal et al., Nature 383:802-804 (1996), which is hereby incorporated by reference in its entirety). Therefore, it is expected that the colorimetric sensor agents and quenching agents are useful in high-throughput arrays (i.e., in a multi-well format) for testing ligands for their affinity to known target molecules. Such arrays will be useful in the development and screening of various non-biopolymer ligands.

### EXAMPLES

The following examples are provided to illustrate embodiments of the present invention but are by no means intended to limit its scope.

### Example 1

### Synthesis of CdSe Nanocrystals

Cadmium selenide nanocrystals were prepared using a variation on the methods of Qu et al. (Nano Lett. 1:333-337 (2001), which is hereby incorporated by reference in its entirety).

In a typical experiment, 0.0298 g (0.232 mmol) cadmium oxide, 0.1150 g (0.413 mmol) n-tetradecylphosphonic acid (TDPA), 0.9621 g (3.38 mmol) stearic acid (SA), and 10.9141 g (28.2 mmol) trioctylphosphine oxide (TOPO, 99% purity) are loaded into a 100 mL, three-necked flask. The flask is then purged with nitrogen and heated to 320° C., at which point 1.40 mL of a 0.2 M trioctylphosphine-selenide (TOP-Se) solution (0.28 mmol Se) is injected rapidly. The temperature is lowered to 290° C. for subsequent nanocrystal growth until the desired size is reached. For example, maintaining crystal growth temperatures for up to about 1 min will allow for production of CdSe crystals that are about 2.5 nm or less. In general, the longer dwell time at crystal growth temperatures, the larger the CdSe crystals. For example, 5 min dwell time should yield crystals of about 3 to about 3.5 nm, and 15 min dwell time should yield crystals of about 5 to about 7 nm. (Other conditions, of course, including materials and temperature, will affect the size of the crystals.) After cooling to approximately 75° C., stopping crystal growth, the nanocrystal solution is dispersed in hexane and stored at room temperature until needed.

### Example 2

### Synthesis of ZnS Capped CdSe Nanocrystals (CdSe/ZnS)

CdSe nanocrystals produced in Example 1 are passivated with a semiconducting shell of ZnS using a modification of the procedures given in Dabbousi et al. (J. Phys. Chem. B 101:9463-9475 (1997), which is hereby incorporated by reference in its entirety) and Hines et al. (J. Phys. Chem. 100:468-471 (1996), which is hereby incorporated by reference in its entirety). The modified procedure is as follows:

0.1 mmol of CdSe nanocrystals is precipitated out of a hexane solution with methanol, centrifuged, and then subsequently re-dissolved in 2 ml of hexane. A flask containing 8 g TOPO (trioctylphosphine oxide) is heated to 190° C. under vacuum for one hour and then cooled to 70° C. under nitrogen. At this time, 1 ml of TOP is added into the reaction flask. Following the addition of the TOP, the CdSe-hexane solution is loaded into a syringe and then injected into the reaction flask. The hexane is then immediately pumped off. The reaction vessel is heated to 130° C. and kept in a range of 130° C.-140° C. during the rest of the capping process.

Inside a glovebox purged with nitrogen, 72.3 μl Diethyl Zinc and 149 μl Hexamethyldisilathiane are mixed with 4 ml Trioctylphosphine (TOP) resulting in a clear ZnS precursor solution. The amount of Zn and S precursors are chosen to correspond to 4 monolayers of a ZnS overcoating when the CdSe core is 3-nm in diameter. The ZnS precursor solution is transferred out of the glovebox and added dropwise into the vigorously stirred reaction mixture slowly during a 10-15 minute period.

After the addition of the ZnS precursor solution is complete, the mixture is kept at 130° C. for 30 min, cooled to 110° C., and then is left stirring for two hours. The capped dots are precipitated using methanol, and then are stored in a mixture of hexane and butanol.

### Example 3

### Preparation of Thioctic Acid Derivative of Tetratryptophan ter-Cyclopentane (TWTCP)

In a 25 ml round-bottom flast, thioctic acid (62 mg, 0.30 mmol) was dissolved into 2 ml dry CH₂Cl₂. This solution was then cooled to 0° C. in an ice bath. In parallel, 403 mg TWTCP was suspended in 6 ml dry CH₂Cl₂. 210 microliters Et2N(i-Pr) were added to the TWTCP suspension, causing all material to dissolve and producing an orange solution. This was then added to the solution of thioctic acid, and allowed to cool an additional 5 minutes. 65 mg dicyclohexyl carbodiimide was added, followed 5 minutes later by 4 mg dimethylamino pyridine. The reaction was allowed to warm to room temperature, and stirred for 18 hours. The resultant mixture was filtered through celite. The celite was washed with 50 ml CH₂Cl₂. The combined solution was transferred to a separatory funnel, and washed with 2×50 ml saturated sodium bicarbonate, 2×50 ml water, and 2×50 ml brine. The organic layer remaining was dried over sodium sulfate, filtered, and concentrated in vacuo to provide a light yellow solid (356 mg). LC-Mass spec showed a mixture of starting material, monosubstituted, disubstituted, and trisubstituted products. The mixture was carried to the next step without purification.

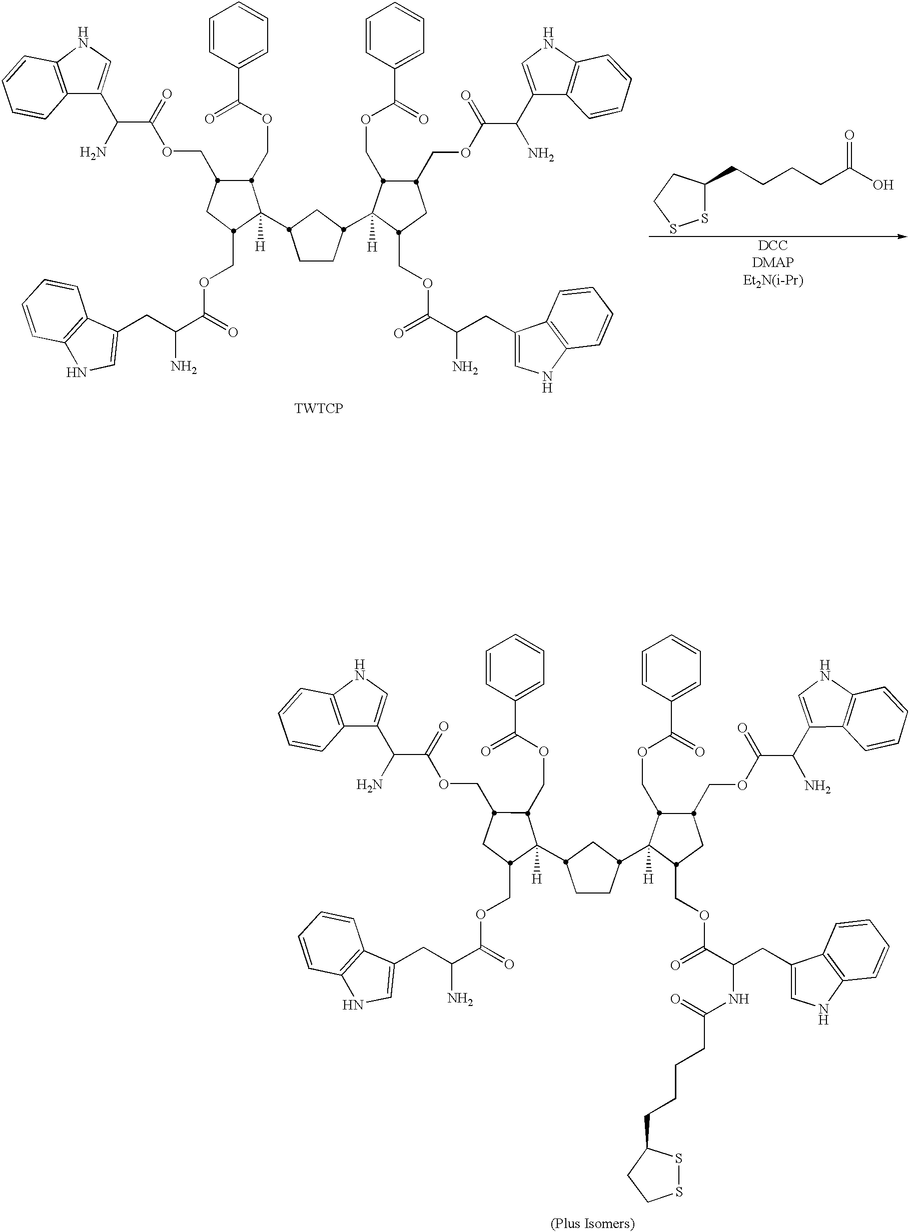

The crude TWTCP-thioctic acid mixture was dissolved into 5 ml dry THF in a 25 ml round-bottom flask. 3 ml absolute ethanol was then added. 180 mg sodium borohydride was then added portion wise as a solid. After 1 hour of additional stirring at room temperature, the solution had completely dissolved. The reaction was allowed to stir an additional 24 hours, then was quenched with 10 ml water. The resultant mixture was diluted with 30 ml ethyl acetate, and transferred to a separatory funnel. The mixture was washed with 3×30 ml water, followed by 1×30 ml brine. The organic layer was then dried over sodium sulfate, filtered, and concentrated in vacuo to provide a white solid.

It has been verified that the TWTCP thiol-derivative retains the ability to bind lipid A with high affinity.

### Example 4

### CdSe/ZnS Nanocrystal Functionalization

CdSe/ZnS core/shell nanocrystals are functionalized with dihydrolipoic acid (DHLA) and the tetratryptophan ter-cyclopentane (TWTCP) derivative, prepared as described in Example 3, using a modified procedure from Mattoussi et al. (J. Am. Chem. Soc. 122:12142-12150 (2000), which is hereby incorporated by reference in its entirety). Approximately 100 mg of CdSe/ZnS core/shell nanocrystals (NCs) in hexane are precipitated with a butanol/methanol mixture and centrifuged. The supernatant is decanted, and the precipitated nanocrystals are re-dissolved in hexane. This procedure is repeated twice more, but the final precipitate is dispersed in 3 mL methylene chloride and transferred to a 25 mL pear-shaped flask containing 100 μL DHLA and 60 mg TWTCP derivative. (The ratio of reactants is 1000:100:1 DHLA:TWTCP:NC). This mixture of NCs, DHLA, and TWTCP derivative is placed under nitrogen, covered with foil, and refluxed in 15 mL methylene chloride at 60° C. overnight. After evaporation of the methylene chloride, the solid is re-suspended in 5 mL dimethylformamide. The acid groups of DHLA are then deprotonated by slow addition of 0.5 g potassium tert-butoxide (approximately five times the amount of nanocrystals used). After stirring at room temperature for 30 minutes, this mixture is centrifuged, the supernatant decanted, and the precipitate dispersed in 10 mL 0.01 M phosphate buffered saline (PBS), pH 7.3. To remove excess potassium tert-butoxide and unbound DHLA and TWTCP derivative, the solution of functionalized nanocrystals is concentrated and rinsed with additional PBS using Millipore Centriplus YM-50 centrifugal filter devices (MWCO 50,000). FIG. 4 illustrates the reaction for TWTCP-functionalization of CdSe nanocrystal particles.

### Example 5

### Construction of Quenching Agent

Although TWTCP binds lipid A with a dissociation constant of 592 nM, it is also able to bind simple sugars like glucosamine with much lower affinity (ca. 20 micromolar). Therefore, binding of a quencher-derivatized sugar to a nanocrystal-immobilized TWTCP should result in quenching of nanocrystal fluorescence, assuming optimal geometry for interaction between the quencher and the nanocrystal (see FIG. 1A).

Gold spheres and rods (prepared using standard techniques by L. Rothberg, University of Rochester) will be used as quenching agents for fluorescence from TWTCP-functionalized CdSe/ZnS nanocrystals. The gold spheres and rods will be functionalized with one or both of the following TWTCP-binding sugar moieties.

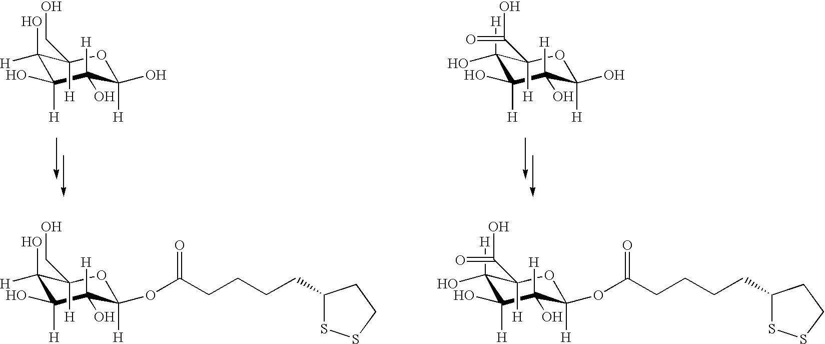

Functionalization of the gold spheres and rods will be carried out by forming a thiol derivative of the above-identified sugar moieties followed by formation of a sulfide bridge between the sugar moieties and the gold spheres and rods. Thioctic acid will be reacted with cyano-imidazole under benzene for 2 hours, followed by reflux conditions to obtain an intermediate compound which is then reacted in NaH/pyridine for six hours and mixed with an appropriate sugar to obtain the compounds shown above. Thereafter, opening of the S-hetero ring can be carried out as described in Example 3 for formation of the thiol (e.g., using NaBH₄ and THF in ethanol).

### Example 6

### Detection of E. coli Using TWTCP-Functionalized CdSe Nanocrystals

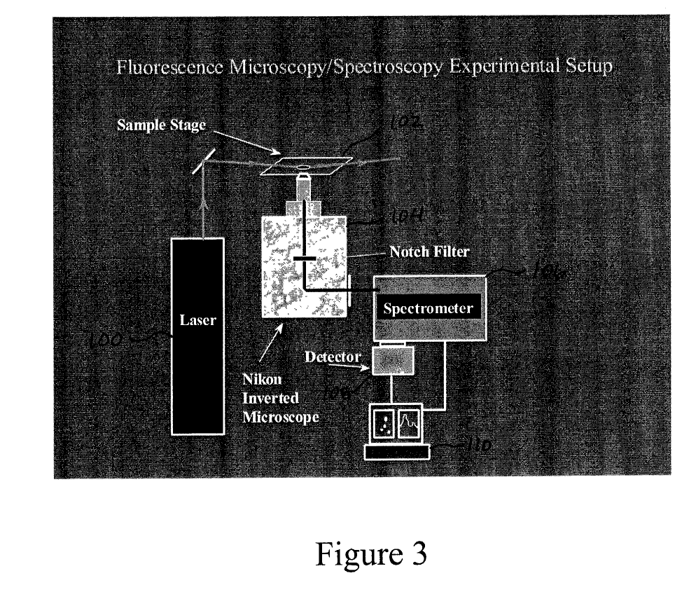

E. coli grown in standard LB media was diluted in PBS until present in an amount equivalent to formation of a monolayer within a drop thereof, centrifuged, and re-suspended in PBS buffer. TWTCP-functionalized CdSe nanocrystals were introduced into the PBS buffer and allowed to incubate for 2 hours (FIG. 5). The solution was placed onto a cover slip, allowed to air dry, and then the cover slip was introduced into the field of a microscope of the type shown in FIG. 3. As shown in FIGS. 6A-B, large aggregates of fluorescent CdSe nanocrystals were observed. It is believed that the TWTCP-functionalized CdSe nanocrystals bound to lipid A on the E. coli. Since a single bacterium has approximately 1000 times the surface area of a nanocrystal, it is expected that thousands of nanocrystals could be bound to a single pathogen.

This experiment will be repeated by filtering the incubated E. coli on a 0.2 μm filter, followed by rinsing 3x in PBS. This should remove any TWTCP-functionalized CdSe nanocrystals that are not bound to E. coli. Thereafter, the bacteria will be re-suspended in water or PBS and examined as described above.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A colorimetric sensor agent comprising:
a nanocrystal particle comprising a semiconductor material and
a non-biopolymer ligand bound to the nanocrystal particle, the non-biopolymer ligand comprising a target molecule-binding moiety and being a peptidomimetic compound, a carbohydrate, or a polypeptide other than an antibody or fragment thereof.

2. The colorimetric sensor according to claim 1, wherein the semiconductor material comprises a group II material, a group III material, a group IV, material, a group V material, a group VI material, or combinations thereof.

3. The colorimetric sensor agent according to claim 2, wherein the semiconductor material comprises a group IV material, a combination of a group IV material and a group VI material, a combination of a group III material and a group V material, or a combination of a group II material and a group VI material.

4. The colorimetric sensor agent according to claim 3, wherein the semiconductor material comprises a group IV material selected from the group consisting of Si and Ge.

5. The colorimetric sensor agent according to claim 3, wherein the semiconductor material comprises a combination of a group IV material and a group VI material, where the group IV material forms a core portion and the group VI material forms a shell portion.

6. The colorimetric sensor agent according to claim 5, wherein the group IV material is Pb and the group VI material is one or more of S, Se, and Te.

7. The colorimetric sensor agent according to claim 3, wherein the semiconductor material comprises a combination of a group III material and a group V material, where the group III material forms a core portion and the group V material forms a shell portion.

8. The colorimetric sensor agent according to claim 7, wherein the group III material is one or more of Ga, In, and Al and the group V material is one or more of N, P, As, and Sb.

9. The colorimetric sensor agent according to claim 3, wherein the semiconductor material comprises a combination of a group II material and a group VI material, where the group II material forms a core portion and the group VI material forms a shell portion.

10. The colorimetric sensor agent according to claim 9, wherein the group II material is one or more of Cd, Zn, and Hg; and the group VI material is one or more of S, Se, and Te.

11. The colorimetric sensor agent according to claim 1, wherein the non-biopolymer ligand comprises a peptidomimetic compound having lipid A-binding activity.

12. The colorimetric sensor agent according to claim 1, wherein the non-biopolymer ligand is covalently bound to the nanocrystal particle.

13. The colorimetric sensor agent according to claim 12, wherein the non-biopolymer ligand is covalently bound to the nanocrystal particle via a sulfide bridge, a phosphine oxide bridge, a carboxylate bridge, or an amido bridge.

14. The colorimetric sensor agent according to claim 1, further comprising a capping molecule covalently bound to the nanocrystal particle.

15. The colorimetric sensor agent according to claim 1, further comprising a polymerization-reactive moiety covalently bound to the nanocrystal particle.

16. In combination,
a colorimetric sensor agent comprising (i) a nanocrystal particle comprising a semiconductor material and (ii) a non-biopolymer ligand bound to the nanocrystal particle, the non-biopolymer ligand comprising a target molecule-binding moiety; and
a quenching agent comprising (i) a metal particle or fluorophore and (ii) a ligand-binding moiety bound to the target molecule-binding moiety of the non-biopolymer ligand, wherein the metal particle or fluorophore absorbs fluorescent emissions from the semiconductor material while the quenching agent remains bound to the colorimetric sensor agent.

17. The combination of claim 16, wherein the ligand-binding moiety binds to the target molecule-binding moiety with an affinity that is lower than an affinity of a target molecule for the target molecule-binding moiety.

18. The combination of claim 16, wherein the quenching agent comprises a metal particle formed of gold, silver, or platinum.

19. The combination of claim 16, wherein the ligand-binding molecule is a sugar.

20. The combination of claim 16, wherein the sugar is glucosamine.

21. A colorimetric sensor agent comprising:
a nanocrystal particle comprising a semiconductor material and a ligand-binding moiety that binds to a target molecule-binding moiety of a non-biopolymer ligand
wherein the nanocrystal particle fluoresces upon dissociation of the ligand-binding moiety from a non-biopolymer ligand that is bound to a substrate that quenches fluorescence thereof.

22. The colorimetric sensor agent according to claim 21, wherein the semiconductor material comprises a group II material, a group III material, a group IV, material, a group V material, a group VI material, or combinations thereof.

23. The colorimetric sensor agent according to claim 22, wherein the semiconductor material comprises a group IV material, a combination of a group IV material and a group VI material, a combination of a group III material and a group V material, or a combination of a group II material and a group VI material.

24. The colorimetric sensor agent according to claim 23, wherein the semiconductor material comprises a group IV material selected from the group consisting of Si and Ge.

25. The colorimetric sensor agent according to claim 23, wherein the semiconductor material comprises a combination of a group IV material and a group VI material, where the group IV material forms a core portion and the group VI material forms a shell portion.

26. The colorimetric sensor agent according to claim 25, wherein the group IV material is Pb and the group VI material is one or more of S, Se, and Te.

27. The colorimetric sensor agent according to claim 23, wherein the semiconductor material comprises a combination of a group III material and a group V material, where the group III material forms a core portion and the group V material forms a shell portion.

28. The colorimetric sensor agent according to claim 27, wherein the group III material is one or more of Ga, In, and Al and the group V material is one or more of N, P, As, and Sb.

29. The colorimetric sensor agent according to claim 23, wherein the semiconductor material comprises a combination of a group II material and a group VI material, where the group II material forms a core portion and the group VI material forms a shell portion.

30. The colorimetric sensor agent according to claim 29, wherein the group II material is one or more of Cd, Zn, and Hg; and the group VI material is one or more of S, Se, and Te.

31. The colorimetric sensor agent according to claim 21, wherein the ligand-binding moiety is a sugar.

32. The colorimetric sensor agent according to claim 31, wherein the sugar is glucosamine.

33. In combination,
a substrate that quenches fluorescence emission,
a non-biopolymer ligand bound to the substrate, the non-biopolymer ligand comprising a target molecule binding moiety, and
a colorimetric sensor agent according to claim 21,
wherein upon dissociation of the ligand-binding moiety from the target molecule-binding moiety of the non-biopolymer ligand, quenching of fluorescent emissions from the semiconductor material by the substrate diminishes.

34. The combination of claim 33, wherein the ligand-binding moiety binds to the target molecule-binding moiety with an affinity that is lower than an affinity of a target molecule for the target molecule-binding moiety.

35. The combination of claim 33, wherein the substrate is formed of gold, silver, or platinum.

36. The combination of claim 33, wherein the ligand-binding molecule is a sugar.

37. The combination of claim 33, wherein the sugar is glucosamine.

38. A substrate having a surface to which is bound a colorimetric sensor according to claim 1.

39. The substrate according to claim 38, wherein the substrate is a solid substrate or a thin film.

40. A method of making a colorimetric sensor agent comprising:
reacting a non-biopolymer ligand comprising a target molecule-binding moiety and a nanocrystal-binding moiety with a nanocrystal particle comprising a semiconductor material or a metal, said reacting being performed under conditions effective to bind the non-biopolymer ligand to the semiconductor material or the metal, thereby forming the colorimetric sensor agent.

41. The method according to claim 40, wherein the conditions are effective to form a covalent bond between the non-biopolymer ligand and the semiconductor material or metal.

42. The method according to claim 40 further comprising:
reacting a capping molecule with the nanocrystal particle under conditions effective to bind the capping molecule to the semiconductor material or metal.

43. The method according to claim 20, wherein said reacting the non-biopolymer ligand and said reacting the capping molecule occur simultaneously.

44. The method according to claim 41, wherein said reacting the non-biopolymer ligand occurs after said reacting the capping molecule.

45. The method according to claim 41, wherein the nanocrystal-binding moiety comprises a thiol group and said reacting comprises formation of a sulfide bridge between the non-biopolymer ligand and the semiconductor material.

46. The method according to claim 41, wherein the nanocrystal-binding moiety comprises an amino group and said reacting comprises formation of an amido bridge between the non-biopolymer ligand and the semiconductor material.

47. The method according to claim 41, wherein the nanocrystal-binding moiety comprises an phosphate group and said reacting comprises formation of an phosphine-oxide bridge between the non-biopolymer ligand and the semiconductor material.

48. The method according to claim 41, wherein the nanocrystal-binding moiety comprises a carboxyl group and said reacting comprises formation of a carboxylate bridge between the non-biopolymer ligand and the semiconductor material.

49. The method according to claim 40 further comprising:
preparing the non-biopolymer ligand by reacting a non-biopolymer precursor comprising a target molecule-binding moiety with a capping molecule comprising a nanocrystal-binding moiety under conditions effective to form the non-biopolymer ligand.

50. A method of making a colorimetric sensor agent according to claim 21, the method comprising:
reacting a compound that includes a ligand-binding moiety and a nanocrystal-binding moiety with a nanocrystal particle that includes a semiconductor material, said reacting being performed under conditions effective to bind the compound to the semiconductor material, thereby forming the colorimetric sensor agent.

51. A method of detecting for the presence of a target molecule in a sample comprising:
introducing a sample to a solution or suspension comprising a plurality of colorimetric sensor agents according to claim 1;
rinsing unbound colorimetric sensor agents from the sample; and
determining whether the rinsed sample fluoresces, indicating the presence of a target molecule in the sample.

52. A method of detecting for the presence of a target molecule in a sample comprising:
introducing a sample to a solution or suspension comprising a plurality of the colorimetric sensor agent-quenching agent combinations according to claim 16;
determining whether the solution or suspension changes color after said introducing, wherein a color or fluorescent emission change indicates the presence of a target molecule in the sample.

53. A method of detecting for the presence of a target molecule in a sample comprising:
introducing a sample to a solution or suspension which includes the combination of the substrate to which is bound the non-biopolymer ligand and the colorimetric sensor agent, in accordance with claim 31; and
determining whether the solution or suspension changes color after said introducing, wherein a color or fluorescent emission change indicates the presence of a target molecule in the sample.

54. A method of detecting for the presence of lipid A in a sample comprising:
introducing a sample to a solution or suspension comprising a plurality of colorimetric sensor agents according to claim 1, wherein the non-biopolymer ligand is a peptidomimetic compound having lipid A binding activity;
rinsing unbound colorimetric sensor agents from the sample; and
determining whether the rinsed sample fluoresces, indicating the presence of lipid A in the sample.

55. A method of detecting for the presence of lipid A in a sample comprising:
introducing a sample to a solution or suspension comprising a plurality of the colorimetric sensor agent-quenching agent combinations according to claim 16, wherein the non-biopolymer ligand is a peptidomimetic compound having lipid A binding activity;
determining whether the solution or suspension changes color after said introducing, wherein a color or fluorescent emission change indicates the presence of lipid A in the sample.

56. A method of detecting for the presence of lipid A in a sample comprising:
introducing a sample to a solution or suspension which includes the combination of the substrate to which is bound the non-biopolymer ligand and the colorimetric sensor agent, in accordance with claim 31, wherein the non-biopolymer ligand is a peptidomimetic compound having lipid A binding activity; and
determining whether the solution or suspension changes color after said introducing, wherein a color or fluorescent emission change indicates the presence of lipid A in the sample.

57. A method of detecting for the presence of Gram negative bacteria in a sample comprising:
introducing a sample to a solution or suspension comprising a plurality of colorimetric sensor agents according to claim 1, wherein the non-biopolymer ligand is a peptidomimetic compound having lipid A binding activity;
rinsing unbound colorimetric sensor agents from the sample; and
determining whether the rinsed sample fluoresces, indicating the presence of lipid A and therefore Gram negative bacteria in the sample.

58. A method of detecting for the presence of Gram negative bacteria in a sample comprising:
introducing a sample to a solution or suspension comprising a plurality of the colorimetric sensor agent-quenching agent combinations according to claim 16, wherein the non-biopolymer ligand is a peptidomimetic compound having lipid A binding activity;
determining whether the solution or suspension changes color after said introducing, wherein a color or fluorescent emission change indicates the presence of lipid A and therefore Gram negative bacteria in the sample.

59. A method of detecting for the presence of Gram negative bacteria in a sample comprising:
introducing a sample to a solution or suspension which includes the combination of the metal substrate to which is bound the non-biopolymer ligand and the colorimetric sensor agent, in accordance with claim 31, wherein the non-biopolymer ligand is a peptidomimetic compound having lipid A binding activity; and
determining whether the solution or suspension changes color after said introducing, wherein a color or fluorescent emission change indicates the presence of lipid A and therefore Gram negative bacteria in the sample.
